(19) United States
(12) Patent Application Publication
(10) Pub. No.: US 2011/0117634 A1
(43) Pub. Date: May 19, 2011

(54) FLAT CELL CARRIERS WITH CELL TRAPS

(76) Inventors: Asaf Halamish, Pardes Chana (IL); Michael Sister, Holon (IL); Lilach Weisz, Tel Aviv (IL)

(21) Appl. No.: 12/988,806

(22) PCT Filed: Apr. 21, 2009
(86) PCT No.: PCT/IL09/00431

§ 371 Date: Feb. 3, 2011

Related U.S. Application Data
(60) Provisional application No. 61/071,276, filed on Apr. 21, 2008.
(60) Provisional application No. 61/129,223, filed on Jun. 12, 2008.
(60) Provisional application No. 61/202,455, filed on Mar. 2, 2009.

(51) Int. Cl. C12M 1/00

(52) U.S. Cl. 435/283.1

(57) ABSTRACT

Cell carrier structures using dynamic flow of cell bearing, washing, or nourishing fluid, from an input reservoir region to an output reservoir region. A common feature of the several structures by which this can be achieved is the presence of channels generally having low height or other cross section, such that the cell bearing fluid readily traverses these channels by capillary action. Optional pumping assistance can also be provided. The capture wells or traps are disposed generally along the length of these channels such that the cells have multiple chances of being captured in a trap or well. The traps or wells are structured such that only a single cell can be trapped in each well or trap, and the disposition of the wells or traps as appendages to the fluid flow channels facilitates the washing or nourishing of the cells while their proliferation or development is being observed.

### FIELD OF THE INVENTION

The present invention relates to the field of cell carriers for use in analytical and bio-analytical methods and more specifically to microfluidic analysis systems, lab-on-a-chip systems, and micro total analysis systems.

### BACKGROUND OF THE INVENTION

Carriers for the analysis of a plurality of individual living cells are known in the art. For example, U.S. Pat. Nos. 4,729,949, 4,772,540, 5,272,081, 5,310,674, 5,506,141, 6,495,340, and co-pending, commonly-assigned PCT application PCT/IB2007/000545, the contents of all of which are incorporated herein by reference, each in its entirety, describe cell carriers comprising grids each having a plurality of holes which are open at both faces of the cell carrier and which are shaped and sized to enable each hole to contain an individual living cell. PCT application PCT/US2006/032355 describes a cell carrier with trapping arrays in a microfluidic format allowing for high density analysis and ease of image processing. Moreover, time-dependent phenomena of a large number of single cells over different time scales are capable of being characterized using this device.

The disclosures of each of the publications mentioned in this section and in other sections of the specification, are hereby incorporated by reference, each in its entirety.

### SUMMARY OF THE INVENTION

The present disclosure describes new cell carrier structures for kinetic observation of individual cells, such as by fluorescence microscopy or other optical methods. The cell carrier separates individual cells, and stores them in separate locations, while maintaining their vitality, such that their development can be viewed over a period of time. The dynamics of the capture of the cells within the cell capture wells or traps is facilitated by the use of assisted flow of the parent cell bearing fluid, from an input reservoir region to an output reservoir region, generally by means of capillary action with or without pumping assistance. A common feature of the several structures by which this can be achieved is the presence of channels generally having low height or other cross section, such that the cell bearing fluid readily traverses these channels by capillary action. The capture wells or traps are disposed generally along the length of these channels such that the cells have multiple chances of being captured in a trap or well. The traps or wells are structured such that only a single cell can be trapped in each well or trap, and the disposition of the wells or traps as appendages to the fluid flow channels facilitates the washing or nourishing of the cells while their proliferation or development is being observed.

A number of different structures are proposed using these common features, each having certain advantages compared to the others. In those implementations where cell capture wells are used, as opposed to lateral traps, the cell capture wells may have one or more openings in their bases, the openings being smaller than the size of the cells to be captured, such that the cell bearing fluid can pass through the openings but not a captured cell. The openings are connected to a common channeling system in fluid communication with a port. The common channeling system should have one dimension, most conveniently its height, sufficiently small that the fluid progresses therealong by capillary action. The capture well entrance dimensions should be of such a size relative to that of the cells to be captured, that only a single cell can enter a well. The pumping port can be used to draw cell-bearing fluid into the cell capture wells by means of capillary action on the whole stream of fluid from the cell captures wells to the pumping port. Pumping can be done either by means of an absorbing medium such as a cotton swab, or positively by means of a pumping pipette.

According to one exemplary variation of such a cell carrier structure, the well can be made in the form of a capture chamber, having lateral dimensions significantly larger than the dimensions of the cells to be captured, such that once a cell has been captured, it can expand, split and flourish within the cell capture chamber. In order to prevent more than a single cell from entering each cell chamber, the outlet opening or openings at the base of the well are arranged in the cell capture chamber to be laterally in the vicinity of the region beneath the well's entrance aperture. So long as pumping sub-pressure is maintained on the outlet opening or openings, a captured cell is held in position beneath the entrance aperture of the well, thereby preventing a second cell from entering the well. Once the required number of wells have been charged with captured cells, the pumping effect can be removed, and the captured cells are then released to move within the cell capture chambers. Because of the lateral dimensions of the cell capture chambers, the cells are able to freely attach, spread and generally proliferate within the well. By this means it becomes possible to capture only a single cell within each cell capture well, and yet to allow that captured cell to flourish within the well when allowed to by freeing it from the bounds of the sub-pressure at the well outlet or outlets.

According to another exemplary implementation of the present claimed invention, the cell carrier may be constructed of an optically transparent carrier base from which a plurality of rows of walls protrudes. A transparent cover is positioned in contact with the upper ends of the walls, such that the regions between adjacent pairs of walls become closed flow channels, whose boundaries are the base, two adjacent walls and the cover. An inlet well enables the cell-bearing host liquid to be applied to one end of the flow channels, through which the liquid flows by capillary action or by the addition of a positively applied pressure difference across the flow channel. An outlet basin at the other end of the flow channels collects superfluous liquid flowing from the channels.

Traps are formed along the length of the walls of the channels, having their openings directed towards the channel, such that as the cell-bearing host liquid flows down the channels, the openings of the traps enable single cells to enter the traps. The trapping walls can have the form of a fishbone-shape, or any adaptation thereof, and the traps are shaped such as to keep the cells captured in the traps, each in its separate trap. In order to enable free flow of the host liquid into the traps, a fluid flow outlet may be provided at the downstream end of the traps. Without such outlets, the fluid within the traps would tend to be static, preventing new fluid from flowing into the traps and depositing a cell. The outlets should be smaller than the inlets, and obviously smaller than the expected trapped cell size, to ensure that the cells are trapped within the traps. According to further implementations, the traps can be formed in the main stream of the channels, away from the walls. According to even further examples of the implementation of the present invention, the structure of the traps and their positioning within the fluid flow down the channels are made to be such that they interfere with stream line flow down the channels, and generate a zig-zag flow pattern down the channels. This can be achieved by staggering the positions of the entrances of the traps down the channels, such that the entrance of one trap is opposite the wall of the trap on the opposite side of the channel. Since the cells have a higher density than that of the parent fluid, the cells tend to be thrown out of the stream where there are changes of direction at the apexes of the zig-zag flow. The positioning of the trap entrances opposite these apexes enhances the likelihood that the cells will be directed into the traps.

In another exemplary implementation of such cell capture structures, similar in some respects to that of the previously described cell carrier, use is made of differential pressure across the cell traps to encourage the flow of cell bearing fluid into the traps, and to keep the cells thus captured in the traps.

According to this implementation, the cell carrier may be constructed of an optically transparent carrier base and cover, between which are disposed multiple rows of double walls. The region between the walls of a set of such double walls defines a first channel. The region between adjacent rows of a set of such double walls defines a second channel, generally larger in cross section than the first channel. Though both of the channels are of such dimensions that flow of the fluid down them can take place by capillary action, suction is added, as will be described below, in order to generate a more positive flow of the fluid. The cover and base are positioned in contact with the ends of the walls, such that both the first channels and the second channels become closed flow channels, whose boundaries are the base, two adjacent walls and the cover. The double walls have protrusions along their length, disposed on that side of the walls such that they project into the second channel, such that they constitute traps to fluid flowing in those second channels. These traps have small openings in their bases, which provide fluid connection between the traps and the first channel on the other sides of the walls of the double walls. The extremities of the second channels open into first and second reservoir regions, where fluid flowing into or out of the second channels can collect. The transparent cover has fluid ports disposed opposite these reservoirs, such that fluid can be input and extracted from one or other of these reservoirs. The first channels are sealed at one end, and open into well structures at their other end. The transparent cover has a manifold channel built into it opposite the region of the enclosed well structures, and a third port in fluid connection with the manifold channel. This entire structure is sealed except for the port openings.

In use, cell-bearing host liquid is applied to one of the first or second ports, such that it flows into the reservoir below, and from there into the second channels. Suction is applied to the third port, and, because of the orifices in the bases of the traps, sucks fluid from the first set of channels, through the traps and into the second set of channels. The orifices are selected to be of such a size that they allow ready flow of fluid through, but are too small to allow passage of the targeted cells in the fluid. Such cells are thus trapped in the traps, where they can be observed by such methods as normal or fluorescence microscopy. So long as the suction is applied, the cells are trapped by the Venturi effect of the fluid flow. Ultimately, they become lodged within the traps, such that they remain there even after the suction has stopped.

Once the cells have been captured, their behavior under the effect of various reagents or drugs can be observed by flowing the reagent or drug down the channels, In particular for the last two implementations, and over the cells. The traps should be of a size such that each trap contains only a single cell, but should be large enough to still provide sufficient room for cell spread and division, with the channel itself providing additional room for spread if required. The cells can also be manipulated if the base or cover is made removable, to gain physical access to the cells.

One exemplary implementation of a cell carrier for capturing cells, as described in this disclosure, comprises:
(i) a planar body member in which is formed an array of cell capturing wells, each of the cell capture wells generally comprising an entrance aperture open to one surface of the body member and a plurality of openings at the end of the well distant from the entrance aperture, the entrance aperture having dimensions relative to that of the cells to be captured, such that only a single cell at a time can enter a well,
(ii) a fluid collection passage in fluid communication with the plurality of openings of the cell capturing wells, and
(iii) a pumping port in fluid communication the fluid collection passage,
wherein the openings have cross sectional dimensions significantly smaller than those of the entrance apertures. In such a device, the openings may be such that a cell of size that the cell capturing well is adapted to capture, cannot pass through them.

In other implementations of such cell carriers, the plurality of openings at the end of the well may be disposed at least partly in the well wall, such that not all of the openings can be simultaneously blocked by the presence of a captured cell. Alternatively, the plurality of openings at the end of the well may be disposed off the axis of the well, such that not all of the openings can be simultaneously blocked by the presence of a captured cell.

In other exemplary implementations of the above described cell carriers, the height of the fluid collection passage may be sufficiently small that fluid disposed in the pumping port flows through the fluid collection passage by capillary action. In this case, the plurality of openings enables fluid flowing through the fluid collection passage to rise into the cell capture wells.

Any of these above-described devices may further comprise a fluid application region in fluid contact with the one surface of the body member, such that fluid deposited in the fluid application region may access the entrance apertures of the capture wells. In such examples, the cell carrier may further comprise a cover positioned on the cell carrier covering the fluid application area and the one surface of the body member, such that fluid applied to the fluid application area flows by capillary action to the one surface of the body member.

Yet other exemplary implementations perform a method of utilizing a cell carrier such as those described in the preceding paragraphs, the method comprising the steps of:
(i) applying a fluid to the pumping port, such that it flows along the fluid collection passage and through at least some of the plurality of openings into the cell capture wells,
(ii) applying a cell bearing fluid such that it reaches the entrance apertures of the cell capture wells, and
(iii) pumping fluid from the pumping port, such that the cell bearing fluid flows into the cell capture wells.

Such an exemplary method may further comprise the steps of:
(iv) applying a washing fluid to the entrance apertures of the cell capture wells, and
(v) pumping fluid from the pumping port, such that the washing fluid is drawn over any cells captured in the cell capture wells.

Another example implementation can involve a cell carrier for capturing cells, comprising:
(i) a planar body member in which is formed an array of cell capture chambers, at least one of the cell capture chambers comprising:
   (a) an entrance aperture open to one surface of the planar body member defining the top end of the at least one cell capture chamber, and disposed at one lateral end of the at least one cell capture chamber, the entrance aperture having cross sectional dimensions which are smaller than those of the at least one cell capture chamber, and which, relative to the dimensions of the cells to be captured, are such that only a single cell at a time can enter the entrance aperture, and
   (b) at least one opening at the bottom end of the at least one cell capture chamber, and positioned at least partly in a wall of the at least one cell capture chamber in the region beneath the entrance aperture,
(ii) a fluid collection passage in fluid communication with the at least one opening of the at least one cell capture chamber, and
(iii) a pumping port in fluid communication the fluid collection passage,
wherein each of the at least one opening has dimensions significantly smaller than those of the entrance apertures.

In such an example implementation, the at least one opening may be such that a cell of size that the cell capturing well is adapted to capture, cannot pass therethrough. Additionally, the height of the fluid collection passage may be sufficiently small that fluid disposed in the pumping port flows through the fluid collection passage by capillary action.

In any of these cell carriers with a cell capture chamber, application of pumping action to the pumping port may be operative to hold a captured cell at the at least one opening at the bottom end of the at least one cell capture chamber, such that a second cell of similar size cannot enter the entrance aperture into the at least one cell capture chamber. In such a case, release of pumping action from the pumping port may be operative to release the captured cell such that it can spread laterally within the cell capture chamber.

A further exemplary cell carrier device described herewithin, comprises:
(i) a base from which a plurality of rows of walls protrude,
(ii) a cover disposed in contact with at least some of the ends of the walls distant from the base, such that at least one closed flow channel is formed between an adjacent pair of walls, the base and the cover, and
(iii) at least one inlet in fluid connection with one end of the at least one flow channel and at least one outlet in fluid connection with a second end of the at least one flow channel, such that a fluid applied at the at least one inlet flows along the at least one flow channel to the at least one outlet,
wherein at least one of the flow channels has a plurality of protrusions positioned down its length, such that cell capture traps are formed between the protrusions and the walls.

In such an exemplary cell carrier, the plurality of protrusions may comprise lateral protrusions attached to the walls along their length. Alternatively, the plurality of protrusions may comprise protrusions extending from at least one of the base and the cover, positioned close to the walls along their length. In either of these exemplary cell carriers, the dimensions of the at least one flow channel may be such that the fluid flows along the at least one flow channel by capillary action. Alternatively or additionally, the fluid may flow along the at least one flow channel by means of a pressure differential established between the ends of the at least one flow channel.

Furthermore, in any of the above-described exemplary cell carriers, at least some of the cell traps along the length of the at least one flow channel may have entrance openings aligned to face into the direction from which the fluid flows. In such a case, at least some of the cell traps may have outflow openings at the end opposite to the entrance openings, the outflow openings being smaller in cross section than the entrance openings. These outflow openings are intended to allow a flow of fluid from the at least one flow channel through the at least some cell traps, such that cells borne by the fluid flow are directed into the cell traps. In any of these cases, the outflow openings may have dimensions such that a cell directed into a cell trap and having dimensions such that only a single such cell can enter the cell trap, cannot pass through the outflow openings.

According to further implementations of these exemplary cell carriers, at least some of the cell traps may protrude from the channel walls, or may be disposed in the channels without contact with the walls.

Additionally, at least some of the protrusions may disposed down the at least one flow channel at locations opposite the entrances of cell traps on the opposite side of the at least one flow channel, such that the lateral protrusions encourage entry of cells into the cell traps on the opposite side of the at least one flow channel. As an alternative and advantageous implementation, the protrusions may be positioned such as to generate zig-zag motion of fluid down the at least one flow channel, such that cells having a higher density than the fluid are directed into the traps, while the fluid continues its zig-zag motion down the at least one flow channel.

According to yet another example implementation of the cell carriers of this disclosure, a cell carrier can comprise:
(i) a base plate,
(ii) a cover plate, and
(iii) a cell trapping structure disposed between the base plate and the cover plate, the cell trapping structure comprising a plurality of sets of double walls, each set of double walls defining a first channel between them, and the spaces between neighboring sets of double walls defining a second channel, at least some of the double walls having protrusions disposed along their length on those sides of the walls that project into the second channel, such that the regions between adjacent protrusions constitute cell traps,
wherein the cover plate and the base plate contact at least some of the walls such that closed flow channels are formed therebetween, the cover plate comprising at least one port in fluid connection with a reservoir at one end of at (east some of the second channels, and at least a second port in fluid connection with a reservoir at a second end of at least some of the second channels, and at least a third port in fluid connection with one end of at least some of the first channels, the other ends of which are sealed,
and wherein at least some of the cell traps have orifices at their wall ends, the orifices providing fluid contact between the cell traps and the first channels.

In such an example cell carrier, the application of suction to the third port generates an accompanying suction effect in the cell traps. In this situation, the suction effect may be operative to direct fluid flowing in at least some of the second channels into at least some of the cell traps. At least some cells borne in the fluid flowing in the at least some second channels may then be trapped in some of the cell traps.

In any of these above described example implementations of the cell carriers of this disclosure, the at least one port in the cover plate may operative to input fluid to the second channels and the at least second port in the cover plate may be operative to output fluid from the second channels. In this case, the input and output ports may be used to convey either one of flushing, washing or nourishing fluid to cells trapped in the cell traps.

Furthermore, in these examples, the orifices may have dimensions such that a cell directed into a cell trap and having dimensions such that only a single such cell can enter the cell trap, cannot pass through the orifice.

Additionally, the cell trapping structure may be constructed as an integral part of either one of the cover plate and the base plate, or alternatively, at least one of the cover plate and the base plate may be constructed of a flexible material such that at least one of them can, when the cell carrier is under positive pressure, separate from contact with the cell trapping structure, such that the fluid can flow more readily into the flow channels.

### BRIEF DESCRIPTION OF THE DRAWINGS

The presently claimed invention will be understood and appreciated more fully from the following detailed description, taken in conjunction with the drawings in which:

FIG. 1 is an isometric schematic cutaway view of the cell capturing wells of one exemplary cell carrier grid described in the present application;

FIG. 2 illustrates schematically how the outflow openings at the base of each well are fluidly interconnected, so as to form a thin, flat, maze-like collection chamber;

FIG. 3 a side-elevation cutaway view of the cell carrier grid shown in FIG. 2;

FIG. 4 illustrates a method of manufacturing the body part of the cell carrier grid, so as to generate the well base outflow openings;

FIG. 5A illustrates the cell carrier grid described in FIGS. 1 to 4 built into a complete cell carrier device, while FIG. 5B is a cutaway cross-sectional drawing of the device of FIG. 5A, showing the fluid collection passage connecting the pumping port with the base openings in the cell capture wells;

FIG. 6 illustrates an example of a complete cell carrier device, constructed with the parts shown in FIGS. 5A and 5B;

FIG. 7 illustrates schematically an alternative exemplary cell carrier grid, which provides the cell with room to attach, spread and proliferate after being captured;

FIG. 8 illustrates schematically a side-elevation cutaway view of the cell carrier grid shown in FIG. 7;

FIGS. 9 and 10 illustrate schematically the operation of the cell carrier grid shown in FIGS. 7 and 8;

FIG. 11 illustrates schematically an overall view of the outer structural parts of another exemplary cell carrier as further described in this application;

FIG. 12 illustrates schematically the internal cell trapping structure for use in the cell carrier of FIG. 11;

FIG. 13 illustrates schematically isometric views of the fishbone structure shown in plan view in FIG. 12;

FIG. 14 is a close up view of a part of FIG. 13;

FIG. 15 illustrates schematically a plan view of an alternative structure for the traps of FIGS. 12 to 14, showing trap outflow openings at the downstream ends and staggered positioning of the trap entrances;

FIGS. 16 and 17 illustrate different examples of the trap structure of FIG. 15 with flow outlets and staggered entrances;

FIG. 18 illustrates schematically the internal cell trapping structure of a further example of a cell carrier described in this application;

FIG. 19 is a schematic close up view of the cell trapping structure of FIG. 18 to illustrate the details of the cell traps and their relationship with the flow channels;

FIG. 20 is a schematic illustration of the top cover of the cell carrier;

FIG. 21 illustrates schematically a close up view of the top cover of FIG. 20, with the third port removed;

FIG. 22 is a schematic cut away isometric illustration from the underside of the cover of the cell carrier device; and

FIG. 23 is a schematic cut away isometric illustration of the complete device described in FIGS. 18 to 22.

### DETAILED DESCRIPTION

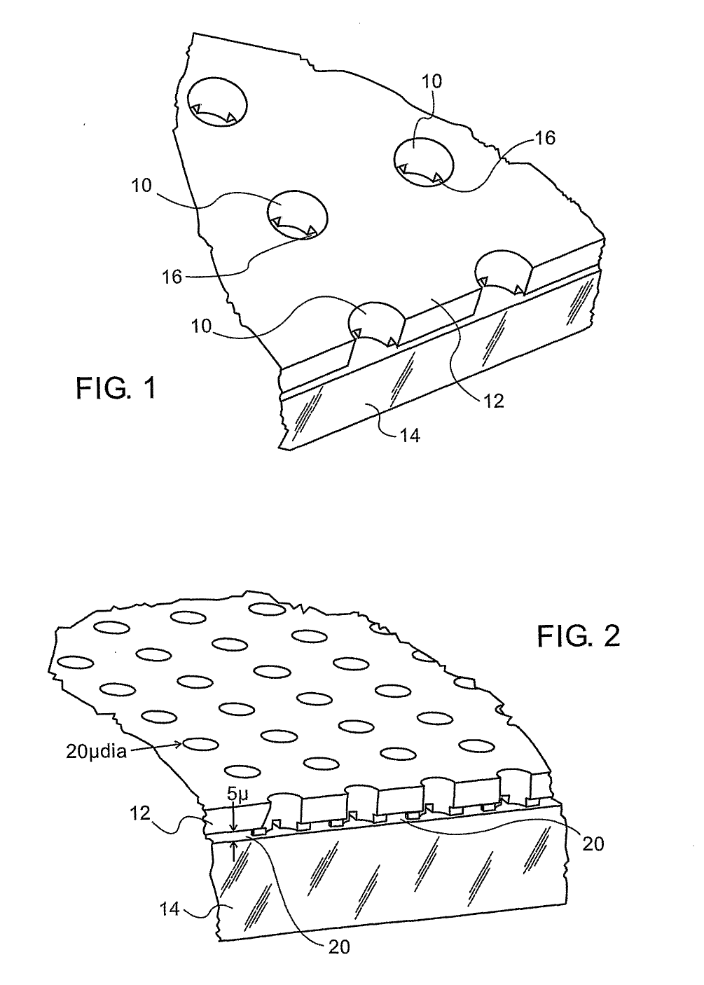

Reference is now made to FIG. 1 which illustrates schematically an isometric cutaway view of the cell capturing wells of an exemplary cell carrier grid of the type described in the present application. The wells 10 are formed within a body layer 12 of generally transparent material, disposed on, and in contact with, a substrate base layer 14, also of a transparent material, such as glass or PMMA. The top of each well has an entrance aperture, and the bottom of each well has one or more outflow openings 16, to enable fluid collected within each well to flow out of the well. It is to be understood that the terms “top” and “bottom” are used throughout this disclosure to indicate respectively, the end of the well with the entrance aperture to which is applied the cell bearing fluid to be observed, and the opposite end of the well. However it is to be understood that the terms are not meant to limit the claimed invention to any particular spatial orientation, and the terms top and bottom are understood to be applicable as described above, regardless of the orientation in which the cell carrier grid is actually held. Furthermore, even though the cell carrier grid is described as though it is manufactured of two separate assembled parts, the body layer and the substrate base layer, it is to be understood that this simply describes a convenient method of manufacture of the cell carrier grid, and is not intended to limit the claimed invention to such a two-part construction. The device could equally well be formed in one piece, for instance in a stereo-lithographic operation.

Reference now made to FIG. 2, which illustrates schematically how the outflow openings at the base of each well are fluidly interconnected, so as to form a thin, flat, maze-like collection passage 20, connecting the bases of essentially all of the wells. Fluid draining from the bottom of the wells thus collects within this flat passage. FIG. 2 also shows how the bottom of the wall of each well may be constructed of a thin shell section with openings to generate the desired well base structure. In FIG. 2 there are also shown suitable dimensions for the construction of the cell carrier. For cell wells of diameter of the order of 20 μm, the height of the thin flat collection passage 20 may be of the order of 5 μm. This height is determined by the height of the thin shell section 40 (as will be seen more clearly in FIG. 4 hereinbelow) with the openings at the base of each well. FIG. 2 also illustrates an advantageous method of construction of the cell carrier grid having a body part 12, conveniently of molded construction, and a flat substrate base layer 14, which can be stuck to the body layer, though it is to be understood that this is not the only method of construction.

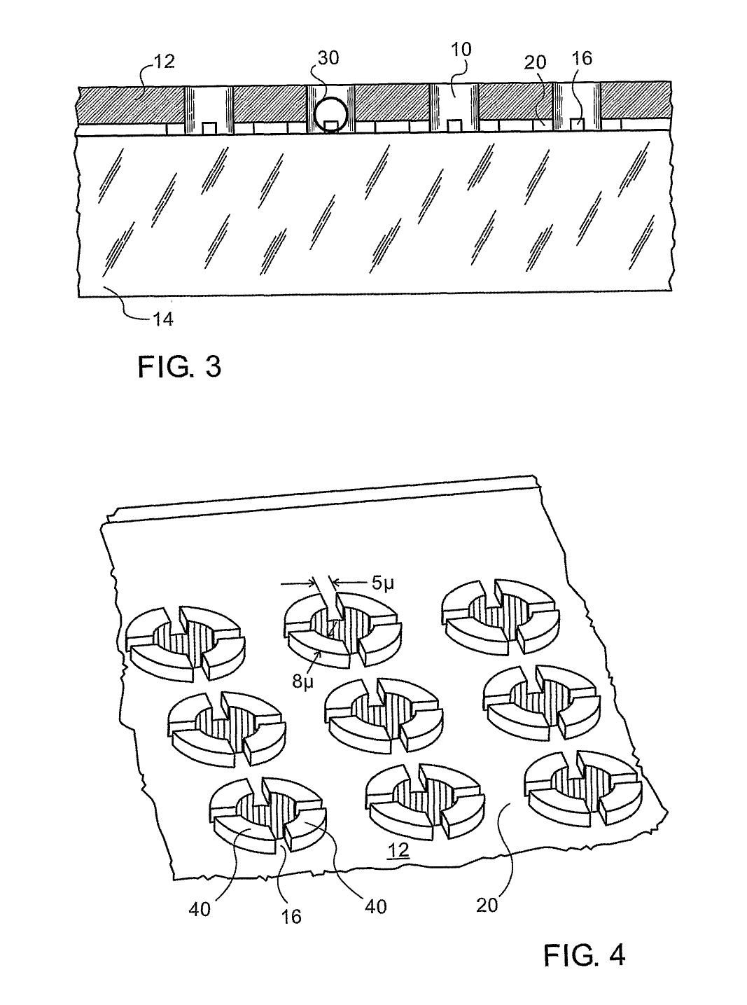

Reference is now made to FIG. 3, which illustrates schematically a side-elevation cutaway view of the cell carrier grid shown in FIG. 2, showing the cell wells 10 with the openings 16 at their bases, connecting to the thin, flat, maze-like collection passage 20. A cell 30 is shown captured in one of the wells. The well diameter may be selected such that only a single cell of the type being observed can be captured in each well. The mechanics of cell capture will be described hereinbelow.

Reference is now made to FIG. 4, which illustrates one convenient method of manufacturing the body part 12 of the cell carrier grid, so as to generate the well base outflow openings 16. FIG. 4 is a view from the bottom side of the body part 12, showing the thin shell like bases of each of the wells 10. The thin, shell-like bases are constructed as feet-like protrusions 40 over an otherwise generally flat surface. When the base substrate layer 14 is attached to the body part 12 in contact with the feet-like protrusions 40, these feet prevent the generally flat surface from coming into contact with the base substrate layer, and generate a space which becomes the thin, flat, maze-like collection passage 20 shown in FIGS. 2 and 3. In the example shown in FIG. 4, the feet-like protrusions 40 have a width of 8 μm and the outflow openings between the feet have dimensions of 8×5 μm.

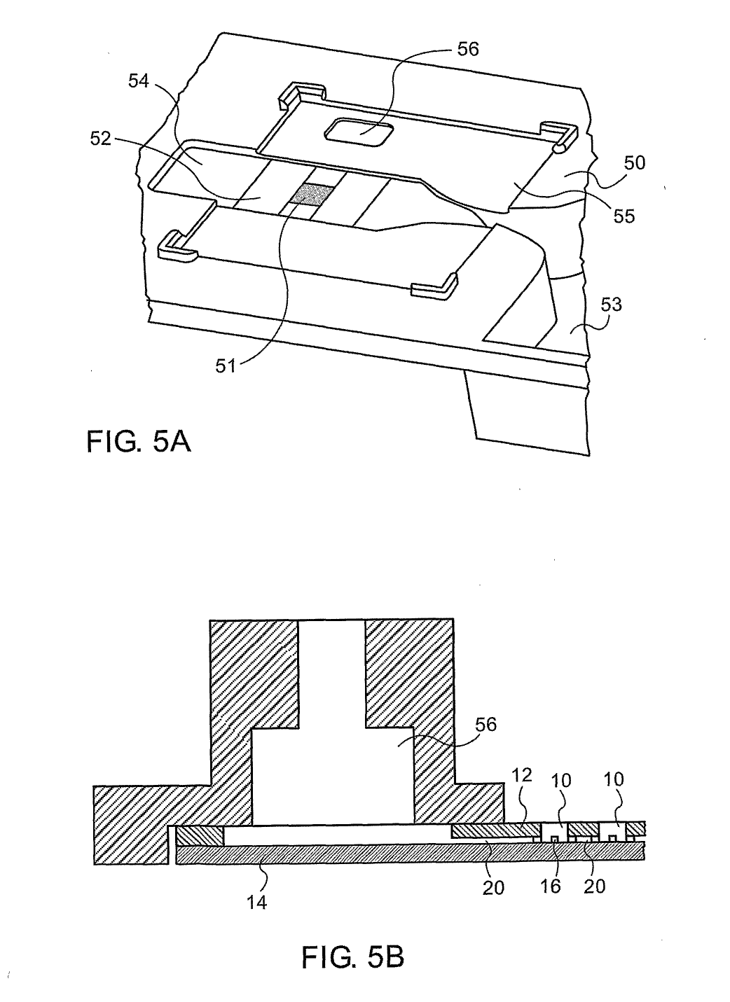

Reference is now made to FIG. 5A which illustrates how the cell carrier grid described in FIGS. 1 to 4 may be built into a complete cell carrier device 50. The grid 51 is mounted in the top surface 52 of the device, onto which the cell bearing fluid is applied, most conveniently through a fluid application opening or niche 54, onto which the cell-bearing fluid can be pipetted. The applied fluid can then run freely over the top surface of the body layer 12 of the grid 51, thus making fluid contact with the entrance apertures of the cell capture wells 10. Beneath the grid, the thin collection passage 20 is in fluid communication with a pumping hole 56 formed in the top cover 55 of the device, such that application of a pumping effect to the hole 56 draws fluid from the thin collection passage 20, and hence ultimately from the cell capture wells 10 in the grid. Conversely, fluid applied at the pumping hole 56, will creep by capillary action to fill the thin collection passage 20, and will tend to rise into the cell capture wells 10. The fluid application opening or niche 54 enables the application of washing fluid for flowing through the grid wells. Fluid overflow from any action is allowed to flow into the drain container 53.

Reference is now made to FIG. 5B, which is a cutaway cross-sectional drawing of the cell carrier device 50 of FIG. 5A, showing how the fluid collection passage 20 connects the pumping port 56 with the base openings 16 in the cell capture wells 10. The low height of the fluid collection passage 20 enables good capillary flow of fluid along the passage.

Referring now back to FIGS. 2, 3 and 5A, the operational advantages and a method of use of this novel cell carrier grid structure will now be explained. According to one exemplary method of use, a few drops of pumping fluid are dripped in to the pumping port 56. Because of the small height of the collection passage, the fluid flows into the collection passage by capillary action. Because of the small dimensions of the openings at the base of the cell collection wells, the fluid may also enter the cell capture wells. The priming of the collection passage with fluid and its entry into the cell capture wells are important to enable the cell bearing fluid to be drawn into the cell capture wells from the top surface of the grid 51. If the cell bearing fluid were simply placed on top of the wells, the surface tension of the fluid may prevent it from entering the wells, and from flowing out of the outlets at the bottom of the wells. Once the wells and the collection passages have been primed, as described above, the cell bearing parent fluid containing the cells to be observed, is now deposited on the top surface of the cell carrier grid 51. A thin glass cover plate can now advantageously be placed over the top surface 52 of the cell carrier device, which has a step located such that the glass cover plate leaves a thin gap between its bottom surface and the grid 51 surface. Fluid dripped into the fluid application region or washing niche 54, will then be continuously drawn onto the grid by capillary action. Application of pumping action at the pumping port 56, using a syringe or pipette, or by means of an absorbent material such as a cotton tipped swab, draws fluid by capillary action out of the thin collection chamber and out of the cell capture wells, thereby pulling the cell bearing fluid down into the capture wells, where some of the cells 30 are captured as shown in FIG. 3. Excess cell bearing fluid can be washed off the top surface of the grid, by use of a washing solution applied in the washing niche 54, flowing over the top of the grid and into the drainage collection container 53, such that only the cells already captured in the wells remain. Furthermore, to enable the cells captured in the wells to be fed while spreading and proliferating, cell nourishing fluid can be applied in the same manner, to the fluid application region or washing niche 54, from where it is drawn to the top surface of the cell carrier grid 51, and drawn into the wells. The excess fluid collects in the drainage collection 53, once a large enough drop of excess fluid is formed to flow down the chute into the drainage collection container, thereby overcoming the tendency of capillary action to keep the fluid from flowing under the effects of gravity, as further explained in commonly-assigned PCT application PCT/IB2007/000545.

Each of the cell capture wells has been shown with four openings to allow passage of fluid into and out of the well. It is to be understood though that use of four openings is only one exemplary implementation, and that the wells could be provided with any convenient number of openings. A single opening may be used, though multiple openings may be preferable, since the captive cell may sit on a single opening and block passage of fluid out of the well. Furthermore the pumping effect may tend to draw a portion of the cell into a single opening, thus applying physical constraints and forces to the captured cell. The use of more than one opening avoids both of these disadvantages.

The cell carrier grid thus allows the capture of a single cell in each of the wells, and cell maintenance in a viable fluid for as long as is necessary to observe cell development, d. The observation can be performed by any of methods known in the art, including fluorescence microscopy. It is to be understood that both the body part and the substrate base of the cell carrier may be made of materials transparent to the light being used for the observation.

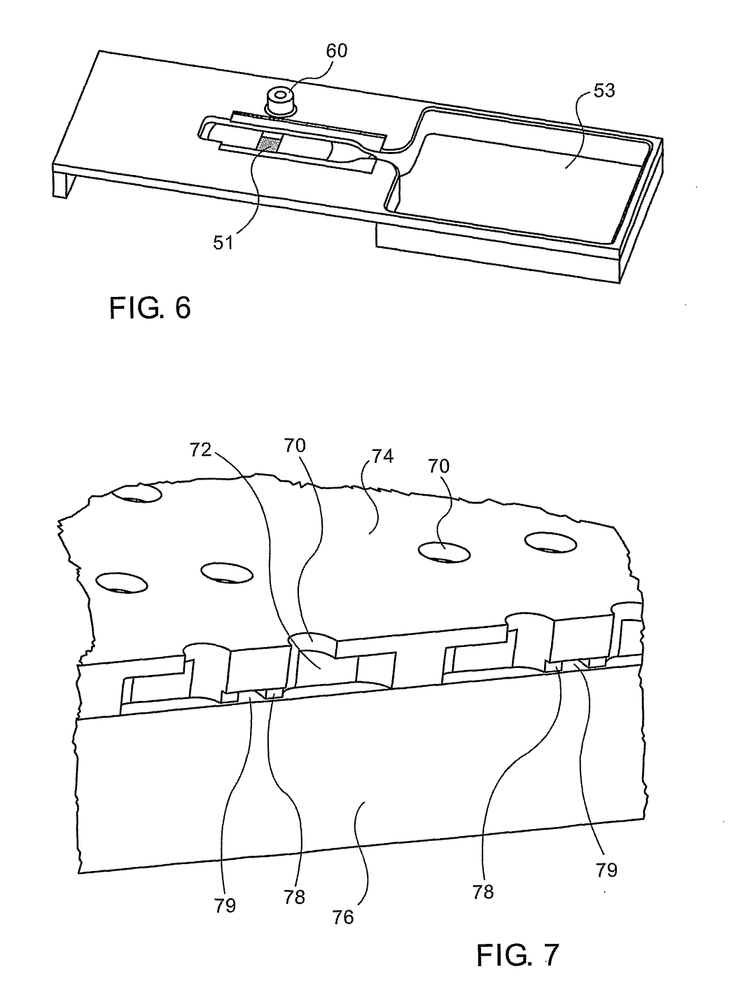

Reference is now made to FIG. 6 which illustrates an example of the complete device, showing the parts mentioned in FIG. 5A, and with a pumping hole 60 designed for taking the tip of a pipette.

The cell carrier described in FIGS. 1 to 6 is particularly suitable for observing single cells, especially non-adherent cells, such as blood cells. However there also exists a need for cell carriers adapted to observe adherent cells, and cells which proliferate and expand during their observation lifetime. In order to accommodate such cells, the capture well must have larger internal dimensions, to enable the cells to spread out while proliferating, spreading or growing. If the well diameter of the grid samples shown in FIGS. 1 to 6 were simply to be made larger to accommodate the increased space requirements, the result may be that more than one cell could possibly enter each well, thereby causing confusion about which cell is being observed, and nullifying the single cell capture advantages of the described structure.

Reference is now made to FIG. 7, which illustrates an alternative exemplary cell carrier grid, which maintains all of the advantages of the structure described in FIGS. 1 to 6, both in construction simplicity and in functional operation, yet which provides the cell with room to expand after being captured, and without letting an additional cell into each well. FIG. 7 is a schematic isometric cutaway view, showing the cell capturing wells of such another exemplary cell carrier grid. Unlike the exemplary devices of FIGS. 1-6, in this example, the capture wells are not straight sided cylindrical, or close to cylindrical openings in the body layer 74 of the device. Instead they are constructed such that the capture well entrances 70 open into a capture chamber 72, larger in dimensions than the diameter of the capture well openings 70. As with the previous example, the diameter of the capture well entrance 70 is adapted to be suitable for capturing a single cell of the type to be observed. The body layer 74 is disposed on, and in contact with a substrate base layer 76, also of a transparent material. Each capture chamber is shown having an opening 78 at its bottom end, to enable fluid collected within it to flow out of the chamber and into a fluid collection channel 79 disposed nearby. The opening 78 at the base of each well is located in a wall position approximately beneath the capture well entrance 70, and in that sector of the projection of the entrance opening generally opposite the opening into the capture chamber 72. The fluid collection channels 79 are all in fluid communication with a collection reservoir, which may be disposed at one side of the cell carrier grid.

Reference now made to FIG. 8, which illustrates schematically a side-elevation cutaway view of the cell carrier grid shown in FIG. 7, showing the cell well entrances 70 in the top surface of the body layer, opening into the capture chambers 72, with the openings 78 at their bases in fluid connection with the fluid collection channels 79. A cell 75 is shown captured beneath the entrance to one of the wells.

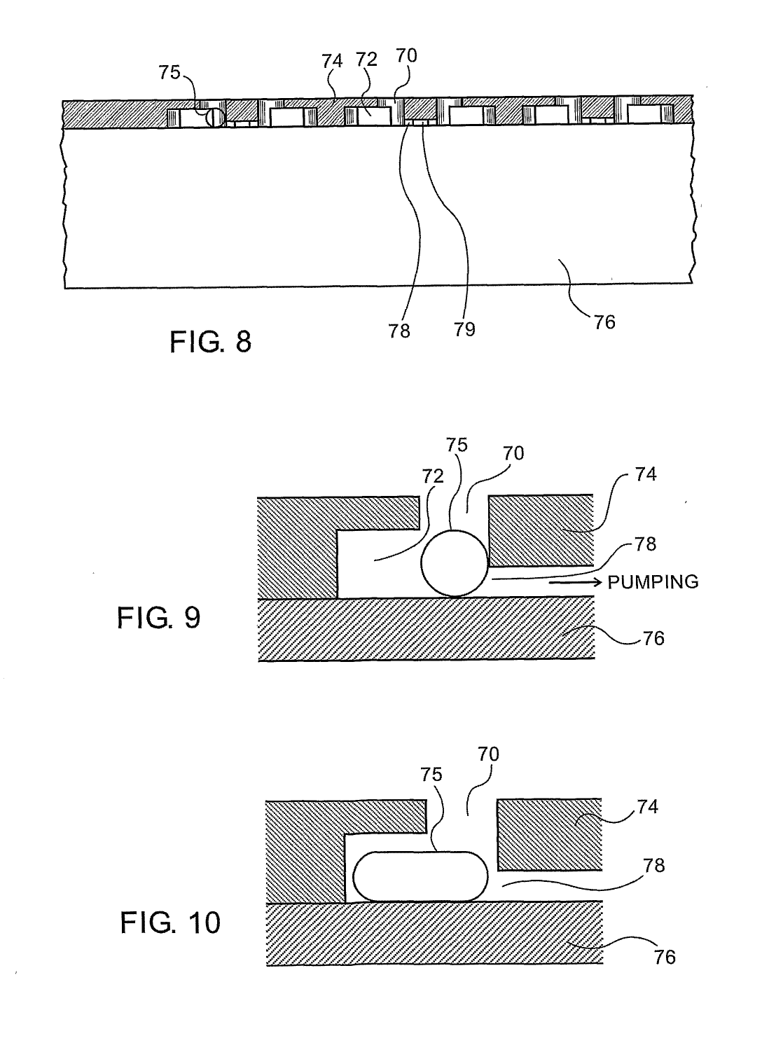

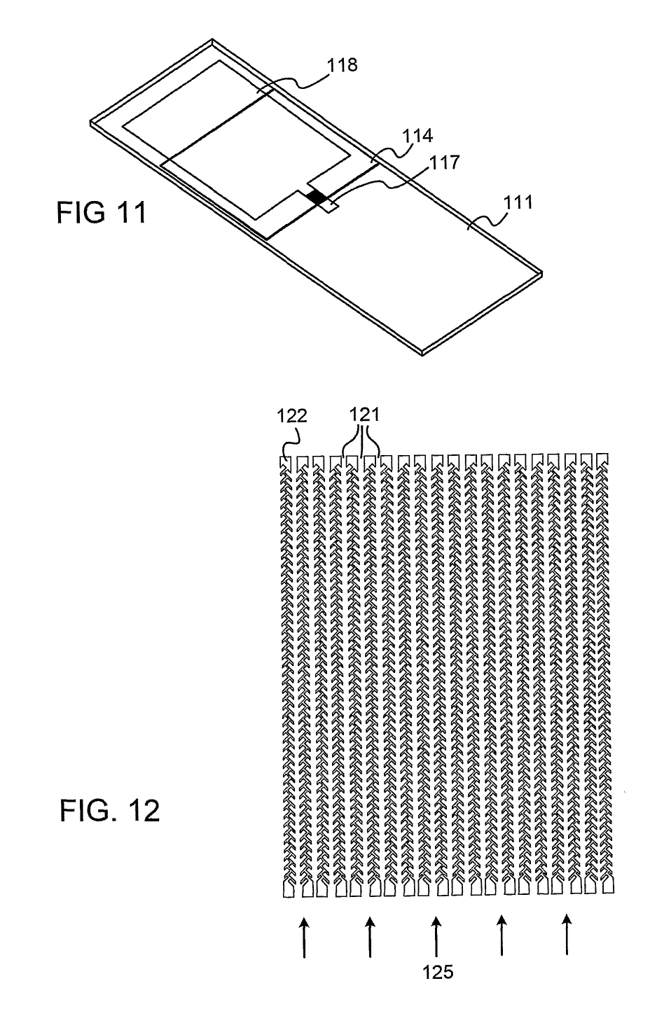

Reference is now made to FIGS. 9 and 10, which illustrates schematically the operation of the type of cell carrier grid shown in FIGS. 7 and 8. The numbering of the items shown in FIGS. 9 and 10 are identical to those used in FIGS. 7 and 8. FIG. 9 shows the cell capture well entrance 70, with a captured cell 75 of diameter slightly smaller than the well entrance. Because of the pumping effect on the well opening 78, the cell 75 is held in position over the opening 78. So long as the pumping negative pressure is maintained, the cell 75 remains in position immediately beneath the capture well entrance, and effectively prevents entry of another cell through the capture well entrance 70. The importance of positioning the opening 78 in the general region beneath the capture well entrance, is now clear, since only in this position will a restrained cell prevents entry of an additional cell into the well entrance 70. Although only one opening 78 is show, it is to be understood that more than one can be used, so long as they are located in the projected region beneath the well entrance, and so long as the effect of the pumping pressure through the openings is sufficient to keep a captured cell restrained at the openings.

Once a sufficient number of cell capture wells have been loaded with cells for observation, the pumping effect can be removed, as explained hereinabove. FIG. 10 now illustrates schematically what happens when this pumping effect is removed. The cell 75 is no longer held to the well base opening 78, and is allowed to move away and expand over the whole of the capture chamber region 72 of the well. Since pumping is stopped only after all of the desired quantity of cells has been loaded, there is now no danger of an additional cell coming and sitting within the cell capture well. At the same time, the size of the capture chamber region 72 is sufficiently large that the captured cell can spread, proliferate and generally thrive without constraint from the cell capture well physical dimensions. Since the well base opening 78 is now clear, fluids such as nourishing or washing fluid can be flowed freely, as required, through the cell capture well.

The limitations as shown in FIGS. 7 to 10, though advantageous for observation of the spreading of adherent cells, is more complex to construct, and does take up more room on the cell carrier grid such that less observation positions per unit area are available.

Reference is now made to FIG. 11, which illustrates schematically an overall view of the outer structural parts of an exemplary cell carrier used for the examples described in this application. The cell carrier has two main parts—a base part 111 for holding the liquid containing the cells to be observed, and a close fitting cover 114, made of a transparent material, such that the cells can be viewed by any of the optical methods used in the art for this purpose. The liquid is inserted into the cell carrier, typically from a pipette, through the filling well 117 in the base, and it flows across the carrier towards the base exit region 118.

Reference is now made to FIG. 12, which illustrates schematically one example of a channel wall structure, which is one feature which differentiates the cell carrier described in this application from prior art cell carriers. The structure is built of a plurality of walls, fishbone shaped in the example shown in FIG. 12, disposed in rows 122, which protrude from the base of the cell carrier and extend up to the height of the cover, thus dividing the internal volume of the carrier into a series of narrow channels 121. These channels run from the filling well 117 region to the exit region 118. The roof of the channels is closed by virtue of the cover 114, which is positioned to be at the same height from the base of the carrier as the height of the walls, such that closed channels are formed. This is another feature which differentiates the cell carrier described in this application from prior art cell carriers. Because of the low height and narrow width of these channels, the liquid containing the cells to be observed may flow through the channels by capillary action. Alternatively, the capillary action may be augmented by means of the positive pumping effect of a pressure difference generated between the filling port and the base exit. This can be simply applied by means of a vacuum pump applied at the base exit 118. In the example shown in FIG. 12, the flow of liquid is indicated by the arrows 125, and is from the bottom of the drawing to the top. Likewise, any consequent reagent or drug medium may be flowed over the cells by means of capillary action or by positive pumping action.

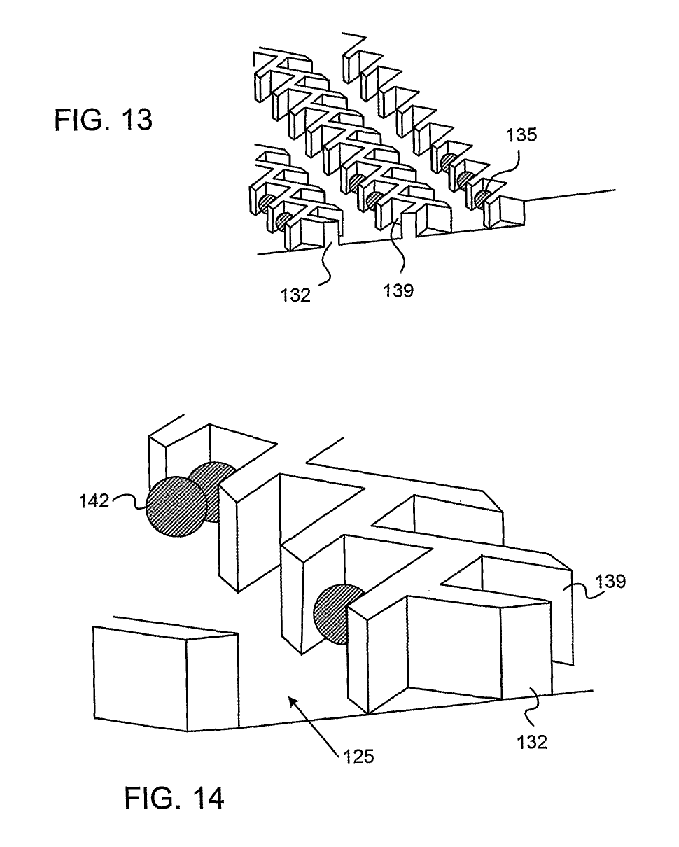

Reference is now made to FIGS. 13 and 14, which illustrate schematically isometric views of a fishbone structure 132 which can be used as one exemplary implementation of the cell traps described in this application. FIG. 14 is a close up view of a part of FIG. 13. Though the traps in FIGS. 13 and 14 are shown as having straight walls, it is to be understood that they can be of any shape, curved, spherical, elliptic, without limiting the scope of the cell carrier described in this application. A feature common to all of these implementations is the generation of closed channels down the length of the cell carrier, with a series of traps 139 in the form of niches or alcoves in the walls of these channels, such that cells contained in the liquid passing down these channels have a high chance of being retained in one of the traps on their way down the channel. The openings of the traps may be at least partly aligned to face the oncoming liquid flow, in order to assist in this trapping action. Furthermore, the traps may be shaped such that once a cell has entered a trap, it is not readily displaced therefrom by the regular flow of liquid around the entrance of the trap. This property can be enhanced if the walls of the trap facing the flow direction are constructed with hollows to allow the trapped cells to be more firmly lodged within the traps. The size of the traps may be such that only one cell can be lodged therein, and once the trap is thus filled, further cells will continue traveling down the channel until they reach a vacant trap in which they may lodge, if the current flow at the entrance to that trap randomly directs the cell into that trap. In the example shown in FIG. 13, the first few traps are shown occupied with cells 135.

Once the entire sample has been inserted into the cell carrier, the channels can be washed with a cell free biological medium, for instance, to sweep out any untrapped cells, and to leave only the trapped cells for analysis. Each trap has its own unique address, such that each cell has its own label which can be used to correlate the results of microscopic observations of individual cell behavior as a function of time thereafter, following activation of the cells by various reagents. This activation can be performed for the entire cell carrier occupants, or it can be varied from channel to channel, such that comparative behavior can be studied between the cells trapped in different channels, according to the reagent flowed through those channels. By this means, the effect of several different reagents or drugs can be observed simultaneously on the cells in one or more different channels. Such embodiments may be implemented by providing separate input wells for different channels or groups of channels. By this means, different channels can also be filled with different cell host liquids.

The cell carrier example, a part of which is shown in FIG. 13, may have 25 channels, each of which may contain 50 traps on either side of the channel, such that a total of 2,500 cells can be studied simultaneously. The size of such an exemplary cell carrier may be 2 mm×2 mm, and the channel width may be of the order of 30 microns, such that the size of each trap is of the order of 20×20 microns, though this can be selected according to the cell types to be observed. Each trap is intended to contain one cell only, though there should be room in the trap itself for some extent of cell spreading, and when this space has been fully used, there is room in the channel itself for the cell to expand, and even to perform cell division, the additional cell being accommodated sticking out into the channel, as shown by exemplary cell 142 in FIG. 14.

The cell carrier body may be made by standard photo-lithographical methods, as is known in the art, and may be made of materials such as PMDA, PMMS, SUB, Polystyrene, Polycarbonate and the like.

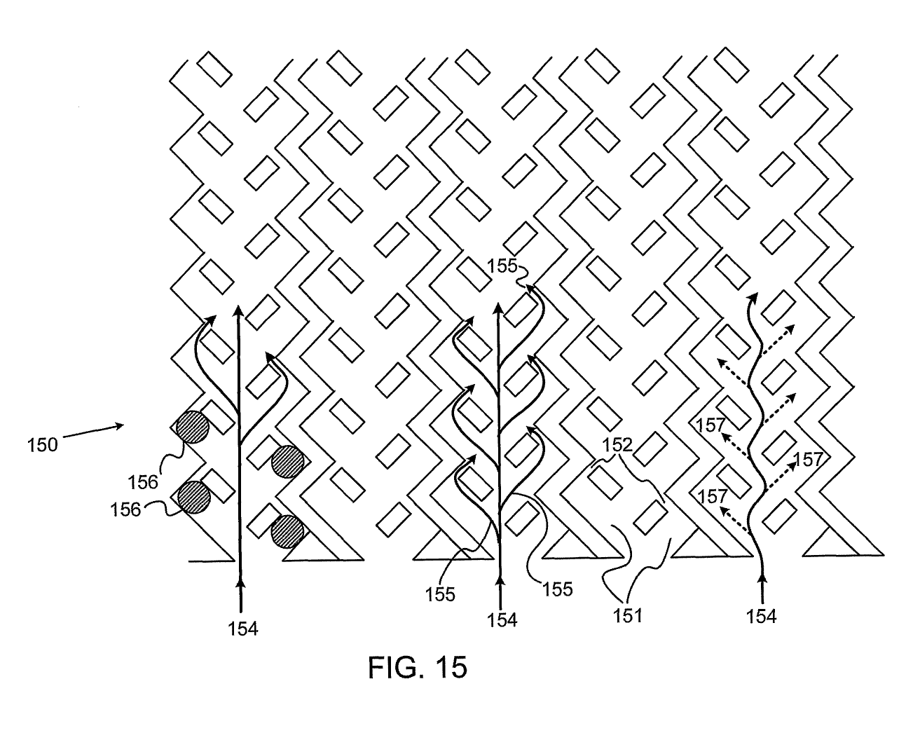

As previously mentioned, the closed trap described in the examples of FIGS. 12 to 14 above may not enable cells to enter the trap freely. Reference is now made to FIG. 15, which illustrates schematically a plan view of an alternative structure 150 for the fishbone trap of FIGS. 12 to 14, in which the traps 151 are provided with outflow openings 152 at their downstream ends in order to enable a free flow of fluid through the trap. Thus, the main flow of fluid 154 enters the structure and passes down the center of the channels, as in the previously described example. Besides the main stream 154 passing down the center of the channel, part of the flow 155 passes into the traps and out of the outflow openings at their downstream ends. The flow through the traps enables cells to enter the traps freely, and the traps fill up gradually with trapped cells 156, as previously described. Additionally, the cells are even directed to enter the traps because of the zig-zag nature of the main flow of fluid, as illustrated in the right hand channel of FIG. 15. The flow in the different channels of the example of FIG. 15 is illustrated in a different manner only in order to demonstrate different advantages of the various aspects of the present invention, but the flow should in fact be the same in all of the channels. Since the cells have a higher density than the fluid, they are less readily able to negotiate the zig-zag path of the main stream, and thus at every change of course, they have more of a tendency to continue along their motion path, to be thrown out of the main stream, and thus to enter the traps, as shown by the dotted arrows 157 in the right hand channel of FIG. 15. This meandering flow effect can be achieved simply by arranging the trap entrances to be positioned opposite trap walls on the opposite side of the channel. It is to be emphasized that this structure is described and claimed in this application to be operable, independently of the actual fluid mechanism by which the cells are encouraged to enter and be trapped by the traps.

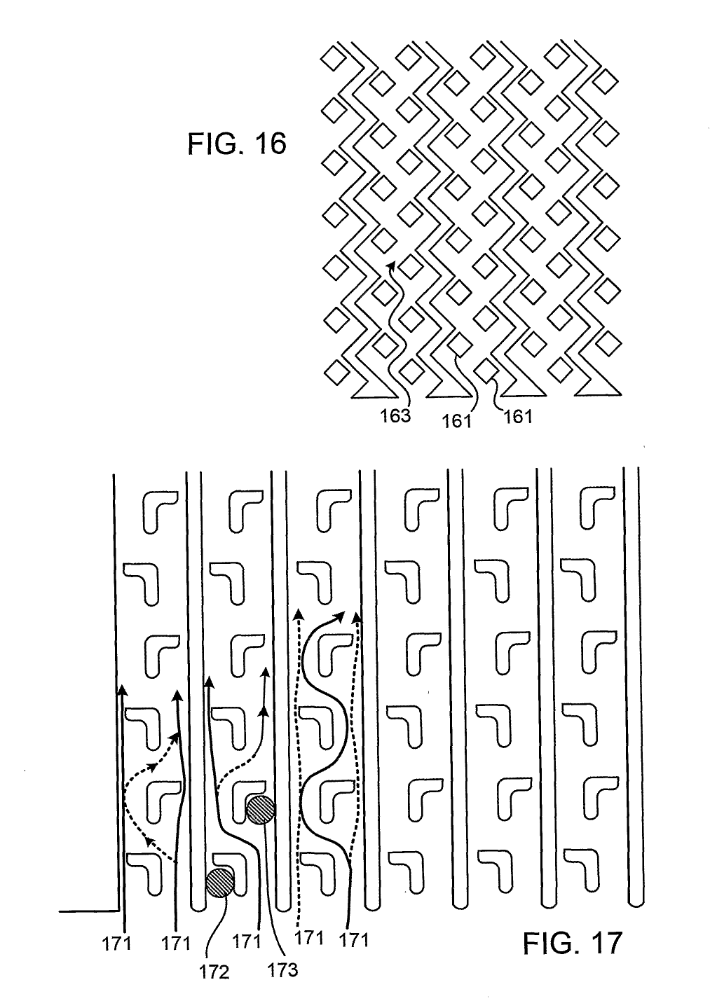

Reference is now made to FIGS. 16 and 17 which illustrate slightly different examples of the structure of FIG. 15. In the exemplary structure of FIG. 16, the projections of the walls 161 of the traps into the main stream of the flow channels is made even more pronounced, such that there is no direct “line of sight” down the channels. This creates an even stronger zig-zag path effect 163 than the exemplary structure of FIG. 15. The width of the channels and the width of the traps in any of the examples shown in this disclosure, and even the height of the channels themselves, can be selected to be larger or smaller in order to suit the size of the cells it is intended to trap.

In the example of the structure of FIG. 17, the trap walls are shaped such that the main fluid stream 171 is split to flow partly down the sides of the channels, and with a likelihood of turbulent cross-over and zig-zag flow path in the center of the channel, as shown by the dotted stream lines in the left-most channel of this example. As the first encountered traps get filled by trapped cells 172, 173, the flow can no longer pass along the sides of the channel, but is forced to negotiate only a zig-zag path around the traps, until it reaches an empty trap downstream, where both the side flow which can again take place, and the zig-zag path, encourage the trapping of a cell. This structure is thus exemplified by a highly meandering path of fluid flow down the center of the channel, combined with a trapping geometry which enhances this meandering effect as the traps are filled, thereby increasing the likelihood of the filling of the next vacant trap by a cell. The ratio of the side flow effect and the tendency for a meandering flow even before any traps have been filled, is determined by the geometric ratio of the openings at the end of each trap and the cross section for flow around the traps. This is illustrated in the third channel from the left in the example of FIG. 17, where the main stream shown as a full line is the meandering path, with a lesser stream, as indicated by the dashed path, flowing down the sides of the channels. However, it is to be emphasized that the structures are described and claimed in this application to be operable, independently of the actual fluid mechanism by which the cells are encouraged to enter and be trapped by the traps.

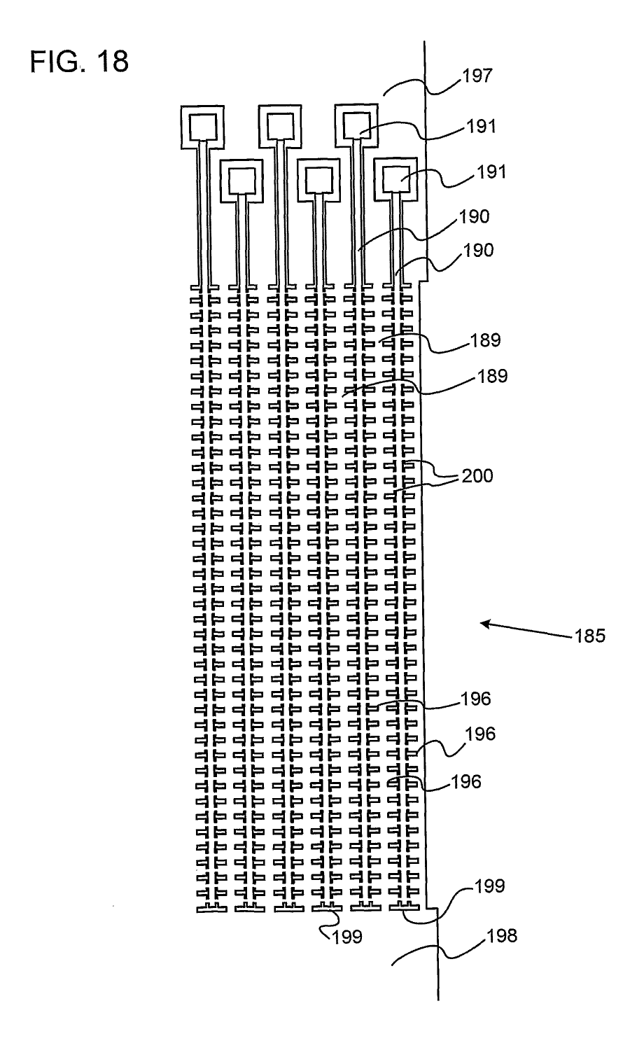

Reference is now made to FIG. 18, which illustrates schematically, an additional exemplary form of cell trapping structure 185 for use in this further described type of cell carrier device. The cell trapping structure comprises an array of double rows of walls 200, each set of double walls having a hollow channel 190 between them. The spaces between each row of double walls define a second set of channels 189 known as the broad channels, and having a generally larger internal flow cross section than that of the so-called hollow channels 190 between the double walls. The broad channels 189 are open at both of their ends, one set of ends into one reservoir region 198, and the other ends into a second reservoir region 197. The channels between the double walls are closed off at one end 199, and at the other end, each channel opens into an enclosed well structure 191. The rows of walls 200 have series of trap walls 196 projecting therefrom, such that the trap walls protrude into the broad channels 189.

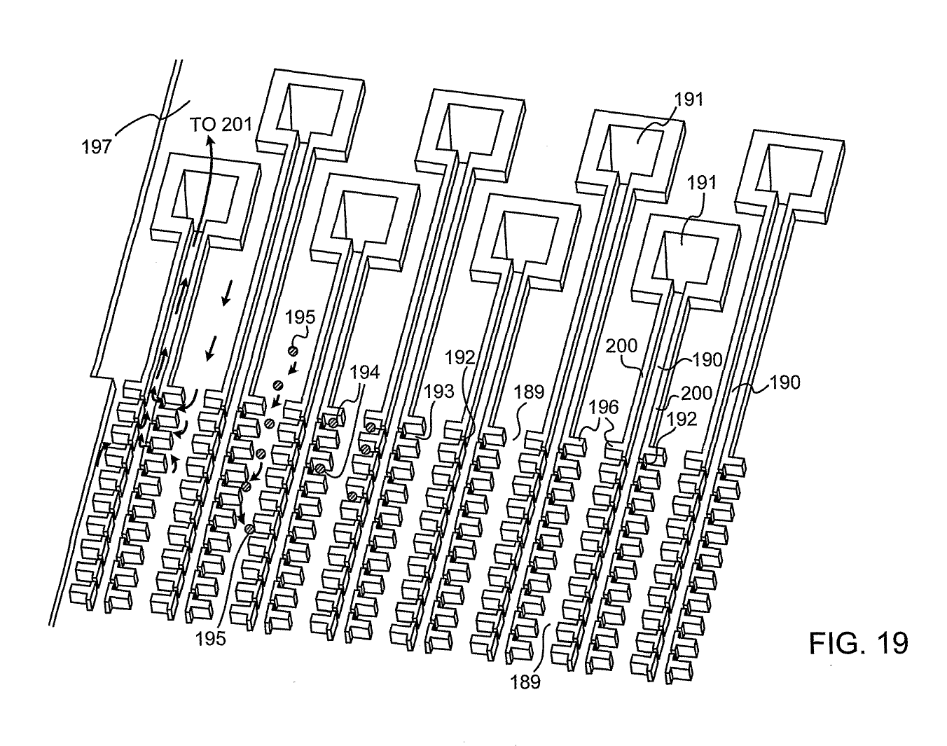

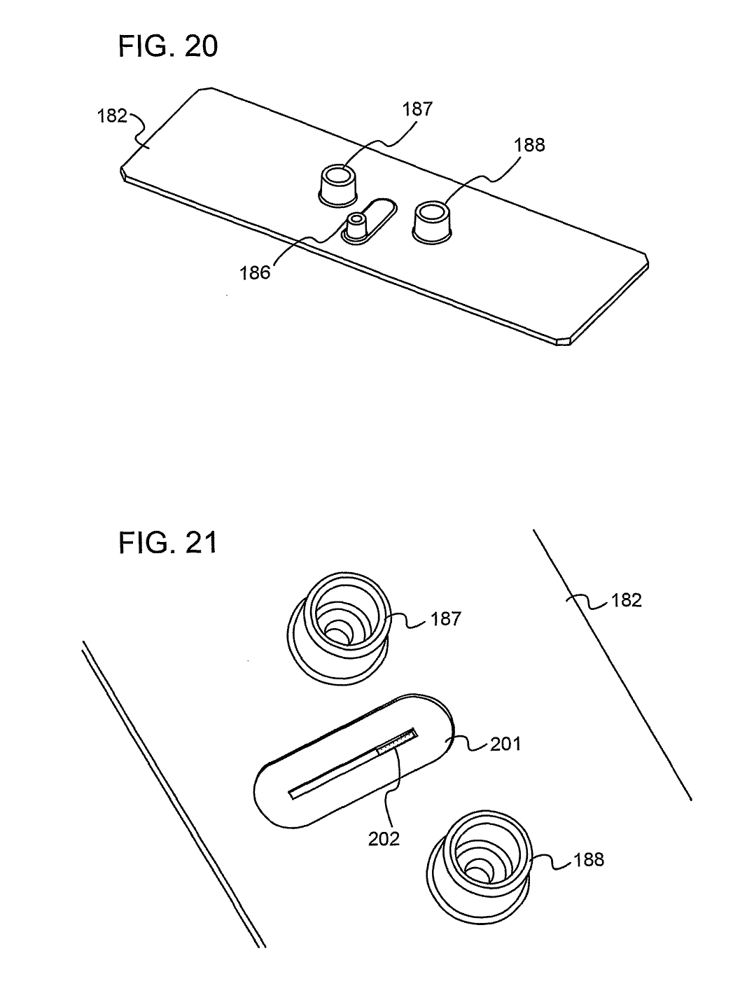

Reference is now made to FIG. 19, which is a schematic close up view of the cell trapping structure 185, to illustrate the details of the cell traps 193 and their relationship with the two sets of channels. Each adjacent pair of trap walls 196 defines a cell trap 193, whose size is generally selected such that it can accommodate a single cell of the type for which that cell structure is intended. In the base of each cell trap 193, there is a small opening 192 through the walls 200 making fluid connection with the hollow channel 190 between each row of double walls 200. Although the trap walls 196 in the example shown in FIGS. 18 and 19 are perpendicular to the rows of double walls 200, this configuration is not intended to limit the invention, but the walls could equally well be inclined at an angle to the perpendicular to the rows of walls 200.

Reference is now made to FIG. 20, which is a schematic illustration of the top cover 182, showing an input and output port 187, 188, which are in connection with the first and second reservoir regions 197, 198, and a third port 186, which is connected to a well channel situated immediately below port 186, the channel being in fluid connection with the well structures 191 when the cover is sealed to the cell capture structure. The use of these ports will be expounded hereinbelow.

FIG. 21 now illustrates schematically a close up view of the top cover 182, with the cover of the third port 186 removed, showing the well channel 201 revealed below the cover, and passageways 202 leading down to the top ends of the well structures sitting in the well channel 201.

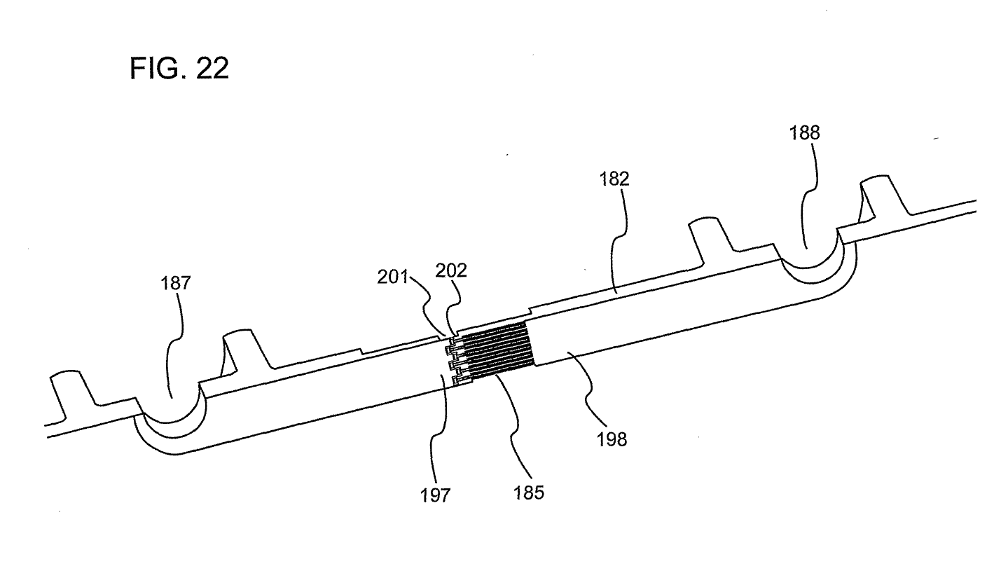

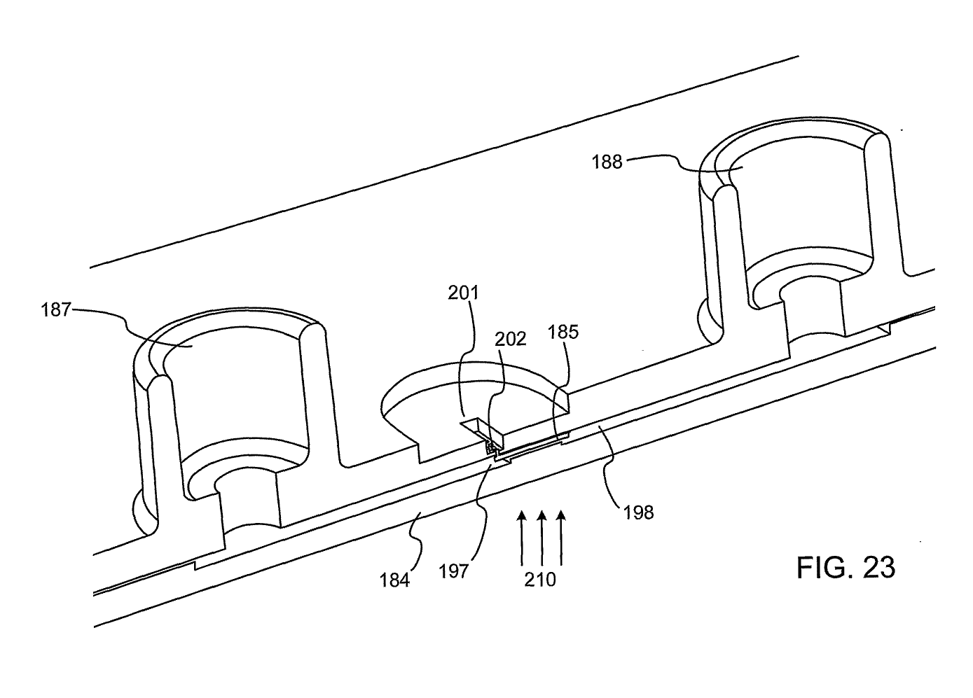

Reference is now made to FIG. 22, which is a schematic cut away isometric illustration from the underside of the cover 182 of the cell carrier device as described in this implementation, to illustrate how the ports are connected to the reservoir areas of the cell trapping structure 185 shown in FIGS. 18 and 19. Port 187 is in fluid connection with reservoir 197 and port 188 is in fluid connection with reservoir 198. The well channel 201 is shown with a series of passageways 202, each connected with an individual well 191. Though not shown in FIG. 22, the suction port 186 is fitted into the top cover 182 over the cell channel 201. The well channel 201 is in fluid isolation from the rest of the structure, except via the passageways 202 and the well structures to the channels 190 between the double walls. The bottom of the structure may be sealed by means of a base plate as shown in FIG. 23 below.

Reference is now made to FIG. 23, which is a schematic cut away isometric illustration of the exemplary cell carrier device described in FIGS. 18 to 22, illustrating how the component parts are assembled into the complete product. The drawing is a view from the top of the cover 182 of the device, showing the input and output ports 187, 188, the well channel 201, and its passageways 202 down to the well structures of the cell trapping structure 185. The transparent base plate 184 is illustrated attached to the cover 182, with small gapped passages provided to enable fluid flow from the ports 187, 188, to the reservoir regions 197, 198, of the cell trapping structure 185. The ends of the base plate are sealed to the cover to make the device fluid-tight. Likewise, the central region of the base plate abuts the bottom of the cell trapping structure 185 to ensure that the channels of the cell trapping structure are sufficiently sealed that they can perform their flow functions correctly. The trapped cells may be viewed in the region of the arrows 210, though it is to be understood that viewing can be from either side of the device.

Referring now to FIGS. 19 and 20 to illustrate the manner in which this exemplary device operates, the ports 187, 188 are used for inputting the cell bearing solution to the device, and for flushing the solution and captured cells away after use. Taking as a non-limiting example that port 187 is the loading port, the cell bearing solution accumulates after entry into the device in the end reservoir 197, from where it flows, generally by capillary action, down the broad channels 189 towards the exit reservoir 198, from where it can be removed through port 188. The fluid can alternatively be sucked into the device by applying suction, such as with a pipette, to port 188. For a cell trapping structure 185 having a symmetrical form, i.e. with its trap walls perpendicular to the length of the channels, it is immaterial which of the ports 187, 188, is the entry port, and which the flushing port. If the traps are asymmetrically constructed, then the ports should be selected such that the flow direction should be that which encourages entry of cells into the asymmetrically aligned traps.

Suction may be applied to port 186, either using a pipette, or a vacuum pump or line, or another vacuum source. As a consequence, the sub-pressure thus generated in the channel 201 is conveyed to the enclosed well structures 191, and thence to the hollow channels 190 between the double wall structures. Since these hollow channels are sealed 199 at their ends remote from the wells, the hollow channels are maintained in a state of sub-pressure relative to the outside environment. This sub-pressure has the effect of sucking fluid passing down the broad channels 189, though the orifices 192 in the walls 200 and into the inter-wall hollow channels 190. These fluid flow lines are shown in one of the rows of FIG. 19 by the set of arrowed lines at the left hand side of the drawing. Since this flow of fluid includes fluid bearing cells 195, as the fluid passes into the traps 193 and through the orifices 192, cells are trapped 194 since the orifices are too small to enable them to pass through.

Once the desired quantity of cells have been trapped 194, the surplus cells still in solution 195 can be flushed out by passing solution down the broad channels 189 from the flushing port to the input port, and the cell carrier is thus left with captured cells ready for inspection and analysis. The cover 182 and the base plate 184 of the device should be constructed of a material which is transparent to the light used to microscopically inspect the cells, and may also be selected to be transparent to any fluorescence emitted by the cells under suitable exciting illumination.

According to another exemplary implementation of the cell carrier shown in this disclosure, the base plate 184 can be assembled in a demountable manner, such that after loading of the cell traps, individual cells can be manipulated microscopically through the base. Additionally, if the base plate is removed, the cells can be readily washed away after inspection.

According to yet another example, the base 184 of the cell carrier may be constructed of an elastic or flexible material, such as a silicone polymer, so that when a positive pressure exists in the channels between the cover and base plate, such as for instance, when fluid is input to one of the ports 187, 188, using positive pressure rather than just dripping it from a pipette or the like, the base plate expands slightly, acquiring a concave shape, thereby enlarging the height of the channels and of the traps. This makes it easier for the cells to get into the channels 189. It may be necessary to put stiffening ribs into the flexible base plate, to prevent it from bulging out too much in the center, away from its attachment points at its periphery.

Though possibly less convenient, the same effects may be obtained if the cover plate is made of an elastic or flexible material.

Use of such a flexible top or bottom plate of the device has a number of possible advantages. The cell carrier is generally constructed such that the channels and traps are marginally higher than the size of the cells to be trapped. Since, however, there is a spread in the size of the cells of any particular type, there may be some cells which will be unable to flow freely into the second channels 189. This expanding base implementation of the cell carrier enables the fluid to flow readily into the channels with greater ease.

Once the cell-bearing fluid is contained within the second (broad) channels, and suction is applied to the suction port 186, the flexible base (or cover) returns to its original position, flush with the base of the walls of the cell trapping structure 185, reducing the height of the channels and traps to their original size, and thus preventing the entry of more than one cell into a single trap. At the same time, the limited height of the traps will tend to keep cells already trapped in their place, either by physical contact with the cell, or, because of the closeness of the trap walls and other boundaries, by preventing the Brownian motion of the fluid around the cell, and thus preventing its flow out of the trap.

After a short time, the trapped cells become temporarily fixed within the traps, and it becomes possible to flow cell nourishing and vitality preserving fluids through the channels. The slight expansion of the base or cover now assists in the flow of this fluid over the entire surface of the cells, without danger that the cells in the traps will be dislodged at this stage. Besides assisting in this perfusion operation, the flexible base or cover also facilitates the application of dyes or other reagents to the trapped cells.

It is appreciated by persons skilled in the art that the present invention is not limited by what has been particularly shown and described hereinabove. Rather the scope of the present invention includes both combinations and subcombinations of various features described hereinabove as well as variations and modifications thereto which would occur to a person of skill in the art upon reading the above description and which are not in the prior art.

We claim:

1. A cell carrier for capturing cells, comprising:
a planar body member in which is formed an array of cell capturing wells, said cell capturing wells generally comprising an entrance aperture open to one surface of said body member and a plurality of openings at the end of said well distant from said entrance aperture and disposed at least partly in the well wall, such that not all of said openings can be simultaneously blocked by the presence of a captured cell, said entrance aperture having dimensions relative to that of the cells to be captured, such that only a single cell at a time can enter a well;
a fluid collection passage in fluid communication with said plurality of openings of said cell capturing wells; and
a pumping port in fluid communication said fluid collection passage,
wherein said openings have cross sectional dimensions significantly smaller than those of said entrance apertures.

2. A cell carrier according to claim 1 wherein said openings are such that a cell of size that said cell capturing well is adapted to capture, cannot pass through said openings.

4. A cell carrier according to claim 1, wherein said plurality of openings at the end of said well are disposed off the axis of said well, such that not all of said openings can be simultaneously blocked by the presence of a captured cell.

5. A cell carrier according to claim 1, wherein the height of said fluid collection passage is sufficiently small that fluid disposed in said pumping port flows through said fluid collection passage by capillary action.

6. A cell carrier according to claim 5, wherein said plurality of openings enables fluid flowing through said fluid collection passage to rise into said cell capture wells.

7. A cell carrier according to claim 1, further comprising a fluid application region in fluid contact with said one surface of said body member, such that fluid deposited in said fluid application region accesses said entrance apertures of said capture wells.

8. A cell carrier according to claim 7, further comprising a cover positioned on said cell carrier covering said fluid application area and said one surface of said body member, such that fluid applied to said fluid application area flows by capillary action to said one surface of said body member.

9-13. (canceled)

14. A cell carrier according to claim 1, wherein application of pumping action to said pumping port is operative to hold a captured cell at said at least one opening at the bottom end of said at least one cell capture chamber, such that a second cell of similar size cannot enter the entrance aperture into said well.

15. A cell carrier according to claim 14, wherein said cell capturing well is widened at its end distant from said entrance aperture, and release of pumping action from said pumping port is operative to release said captured cell such that it can spread laterally within said widened region.

16. A cell carrier, comprising:
a base from which a plurality of rows of walls protrude;
a cover disposed in contact with at least some of the ends of said walls distant from said base, such that at least one closed flow channel is formed between an adjacent pair of walls, said base and said cover; and
at least one inlet in fluid connection with one end of said at least one flow channel and at least one outlet in fluid connection with a second end of said at least one flow channel, such that a fluid applied at said at least one inlet flows along said at least one flow channel to said at least one outlet,
wherein said at least one flow channel has a plurality of protrusions positioned down its length, such that cell capture traps are formed between said protrusions and said walls.

17. A cell carrier according to claim 16 and wherein said plurality of protrusions comprises either lateral protrusions attached to said walls along their length, or protrusions extending from at least one of said base and said cover, positioned close to said walls along their length.

18-20. (canceled)

21. A cell carrier according to claim 16 and wherein at least some of said cell traps along the length of said at least one flow channel have entrance openings aligned to face into the direction from which said fluid flows.

22. A cell carrier according to claim 21 and wherein at least some of said cell traps have outflow openings at the end opposite to said entrance openings, said outflow openings being smaller in cross section than said entrance openings, such that a cell directed into a cell trap cannot pass through its outflow opening.

23. A cell carrier according to claim 21, wherein said outflow openings allow a flow of fluid from said at least one flow channel through said at least some cell traps, such that cells borne by said fluid flow are directed into said cell traps.

24-26. (canceled)

27. A cell carrier according to claim 16, wherein at least some of said protrusions are disposed down said at least one flow channel at locations opposite the entrances of cell traps on the opposite side of said at least one flow channel, such that said lateral protrusions encourage entry of cells into said cell traps on the opposite side of said at least one flow channel.

28. A cell carrier according to claim 16, wherein said protrusions are positioned such as to generate zig-zag motion of fluid down said at least one flow channel, such that cells having a higher density than said fluid are directed into said traps, while said fluid continues its zig-zag motion down said at least one flow channel.

29. A cell carrier, comprising:
a base plate;
a cover plate; and
a cell trapping structure disposed between said base plate and said cover plate, said cell trapping structure comprising:
a plurality of sets of double walls, each set of double walls defining a first channel between them, and the spaces between neighboring sets of double walls defining a second channel, at least some of said double walls having protrusions disposed along their length on those sides of said walls that project into said second channel, such that the regions between adjacent protrusions constitute cell traps;
wherein said cover plate and said base plate contact at least some of said walls such that closed flow channels are formed therebetween, said cover plate comprising at least a first port in fluid connection with a reservoir at one end of at least some of said second channels; and at least a second port in fluid connection with a reservoir at a second end of at least some of said second channels, and at least a third port in fluid connection with one end of at least some of said first channels, the other ends of which are sealed,
and wherein at least some of said cell traps have orifices at their wall ends, said orifices providing fluid contact between said cell traps and said first channels.

30. A cell carrier according to claim 29, wherein the application of suction to said third port generates an accompanying suction effect in said cell traps, directing fluid flowing in at least some of said second channels into at least some of said cell traps, such that at least some cells borne in said fluid flowing in said at least some second channels are trapped in some of said cell traps.

31-32. (canceled)

33. A cell carrier according to claim 29, wherein said at least a first port in said cover plate is operative to input fluid to said second channels and said at least second port in said cover plate is operative to output fluid from said second channels.

34. (canceled)

35. A cell carrier according to claim 29, wherein said orifices have dimensions such that a cell directed into a cell trap and having dimensions such that only a single such cell can enter said cell trap, cannot pass through said orifice.

36. A cell carrier according to claim 29, wherein said cell trapping structure is constructed as an integral part of either one of said cover plate and said base plate.

37. A cell carrier according to claim 29, wherein at least one of said cover plate and said base plate is constructed of a flexible material such that at least one of them can, when said cell carrier is under positive pressure, separate from contact with said cell trapping structure, such that said fluid can flow more readily into said flow channels.
